(19) United States
(12) Patent Application Publication
(10) Pub. No.: US 2026/0341419 A1
(43) Pub. Date: Oct. 29, 2026

(54) INFORMATION PROCESSING DEVICE, INFORMATION PROCESSING METHOD, AND A NON-TRANSITORY COMPUTER READABLE MEDIUM

(71) Applicant: Preferred Networks, Inc., Tokyo-to (JP)

(72) Inventors: Kohei SHINOHARA, Tokyo-to (JP); Takuya SHIBAYAMA, Tokyo-to (JP)

(73) Assignee: Preferred Networks, Inc., Tokyo-to (JP)

(21) Appl. No.: 19/464,756

(22) Filed: Jan. 30, 2026

(30) Foreign Application Priority Data
Jan. 31, 2025 (JP) 2025-015452

(51) Int. Cl. G16C 20/10; G16C 20/70

(52) U.S. Cl. CPC G16C 20/10; G16C 20/70

(57) ABSTRACT

An information processing device includes at least one memory and at least one processor. The at least one processor is configured to obtain one or more first products, evaluate uncertainty of an evaluation value for each of the one or more first products using information from one or more evaluators, and obtain one or more second products based on the evaluation result of the uncertainty.

### CROSS REFERENCE TO THE PRIOR APPLICATIONS

This application is based upon and claims the benefit of priority from Japanese Patent Application No. 2025-015452, filed on Jan. 31, 2025, the entire contents of which are incorporated herein by reference.

### FIELD

The present disclosure relates to an information processing device, an information processing method, and a non-transitory computer readable medium.

### BACKGROUND

Crystal Structure Prediction (CSP) is sometimes realized by generating a large number of crystal structures and predicting energy. In this method, when an accuracy of evaluation (e.g., energy of the crystal structure) for the generated crystal structures is not high, the performance of the search may deteriorate. Eliminating or suppressing this decrease in evaluation accuracy is a necessary configuration for performing CSP calculations.

For example, when executing CSP, it is conceivable to predict whether the accuracy of evaluation of crystal structures generated based on atomic structures will be low, and to exclude samples predicted to have low evaluation accuracy from being used during CSP execution. However, when the number of corresponding elements increases in CSP, it is difficult to select atomic structures for which the accuracy of calculating evaluation values is low, and as a result, there is a problem that the inference of CSP breaks down.

### BRIEF DESCRIPTION OF DRAWINGS

FIGS. 1-3 are diagrams schematically illustrating examples of an information processing system according to an embodiment.

FIG. 4 is a flowchart showing processing of an information processing device according to an embodiment.

FIG. 5 is a block diagram schematically illustrating an information processing device according to an embodiment.

FIGS. 6 and 7 are block diagrams schematically illustrating an information processing system according to an embodiment.

FIGS. 8 and 9 are diagrams schematically illustrating a process flow according to an embodiment.

FIG. 10 is a block diagram schematically illustrating an example of an implementation according to an embodiment.

### DETAILED DESCRIPTION

According to one embodiment, an information processing device includes at least one memory and at least one processor. The at least one processor is configured to obtain one or more first products, evaluate the uncertainty of evaluation values for each of the one or more first products using information from one or more evaluators, and obtain one or more second products based on the evaluation result of the uncertainty.

A problem to be solved by embodiments of the present disclosure is not limited to the problems described above, and as examples of some non-limiting problems, the problem may also correspond to the effects described in the embodiments. That is, the problem corresponding to at least any one of the effects described in the description of the embodiments of the present disclosure can be regarded as a problem to be solved by the present disclosure.

Hereinafter, embodiments of the present disclosure will be described with reference to drawings. The drawings and the description of the embodiments are presented as examples and do not limit the present invention. Further, although the embodiments of the present disclosure are described using a crystal structure for executing CSP, the spirit of the invention of the present disclosure is not limited thereto and can be applied to various applications using crystal structures, atomic structures, and the like.

In the present disclosure, unless otherwise specified, processing may be executed by a processor of an information processing device.

When handling or searching for crystal structures and atomic structures (hereinafter, may be referred to as crystal structures, etc.), including CSP, a plurality of crystal structures, etc. are generated by some method, and searching, etc. can be realized by an appropriate algorithm based on the plurality of generated crystal structures, etc.

For example, the plurality of crystal structures, etc. are generated by some method, structure optimization is performed on the generated crystal structures, etc., evaluation values (e.g., energy) relating to the structure-optimized crystal structures, etc. are obtained, and based on this result, new candidates for crystal structures, etc. can be generated. The present disclosure is not limited thereto, and a configuration in which structures are generated by a diffusion model or the like and evaluation values are obtained may also be employed.

The information processing device can also execute generation of the plurality of crystal structures, etc. by one or more means. In one embodiment, the information processing device generates the plurality of crystal structures, etc. by the one or more means, and when the prediction accuracy of the evaluation values relating to the crystal structures, etc. is considered to be low, determines that the evaluation relating to the crystal structures, etc. is not appropriate, and can execute crystal search by an appropriate algorithm using the crystal structures, etc. from which the crystal structures, etc. are excluded.

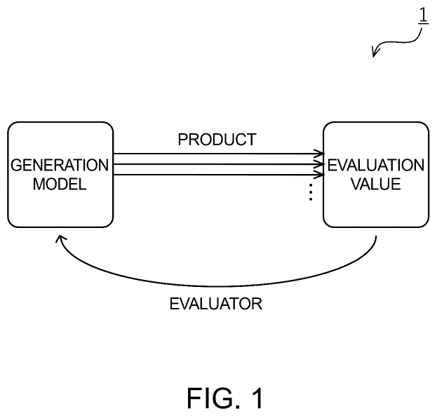

FIG. 1 is a diagram schematically illustrating an example of an information processing system according to an embodiment. The information processing system 1 includes a generation model and an evaluator. The generation model and the evaluator may be implemented by one or more information processing devices.

One or a plurality of generation models may be provided. The one or more generation models (hereinafter, may be simply referred to as the generation model) generate a target product. When a plurality of generation models are provided, each may be a different model. The different models may be models with different parameters, models with different hyperparameters, models formed in different formats, models trained using different training data, or etc. When one generation model is provided, the generation model is not particularly limited in its format, parameters, etc.

The evaluator calculates and outputs an evaluation value for a product obtained by the generation model. In the information processing system 1, the generation model may execute further new generation based on the evaluation value output from this evaluator. The information processing system 1 can realize searching for a target by repeatedly calculating the generation based on the evaluation values and the evaluation of the products. The evaluation may be, for example, some feature quantity of the product. The feature quantity may be a quantity serving as an index indicating the target quality of the product.

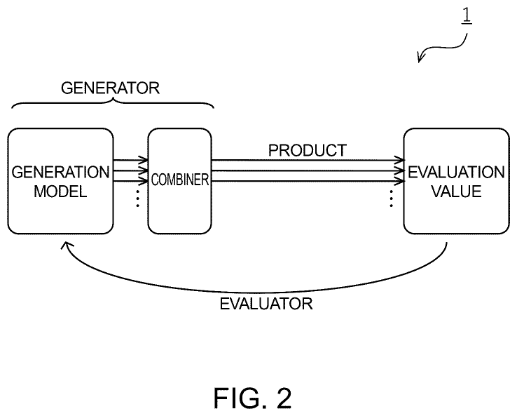

FIG. 2 is a diagram showing another form of the information processing system 1. Outputs of the generation model may be combined by a combiner, and an output of the combiner may be input to the evaluator. The combiner may be a model that generates a plurality of products from a plurality of products. The combiner may be formed based on, for example, a Genetic Algorithm (GA).

As shown in the figure, the plurality of generation models and the combiner may collectively be referred to as a generator. That is, the generator can generate a plurality of types of products using the plurality of generation models.

The information processing system 1 shown in FIG. 1 or FIG. 2 may be, for example, a system that realizes crystal search. In this case, the generation model is a model that generates crystal structures, the combiner is a model that generates a plurality of crystal structures from a plurality of crystal structures, and the evaluator may include a model that obtains physical property values for crystal structures. The physical property values may be, for example, energy, force, etc. of the crystal structure. Of course, as described above, the combiner may be a concept included in the generator, or may not have a combiner at all.

In the information processing system 1, the evaluator executes evaluation of targets generated by the generation model or the combiner, that is, the generator. When the evaluator itself is an evaluation model, this evaluator may perform evaluation depending on training data used in training. The evaluator can, for example, output with relatively high accuracy for data used in training and data in the vicinity of the data used in training, but it is unknown whether it is able to output with high accuracy for data that is distant from the data used in training.

The information processing device in the present disclosure can improve the performance of the generation method by excluding, from generation of targets in the next generation model, products for which there is a possibility that the accuracy of evaluation values is not high in the evaluator.

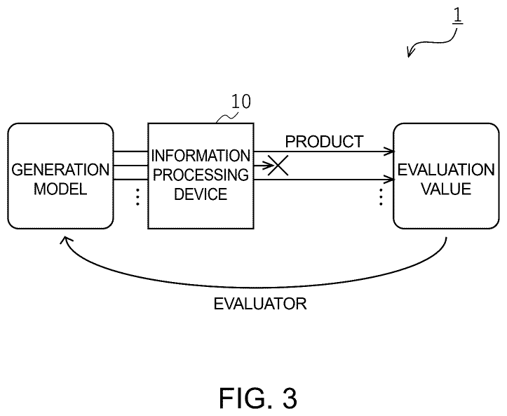

FIG. 3 is a diagram schematically illustrating a non-limiting example of an information processing system 1 including an information processing device according to an embodiment. The information processing system 1 includes an information processing device 10. The information processing device 10 may be at least one information processing device among one or more information processing devices forming a generator or one or more information processing devices forming an evaluator. That is, the information processing device 10 may execute operations of the generator and the evaluator.

The information processing device 10 excludes and outputs products for which accuracy of evaluation in the evaluator is estimated to be not high, regarding outputs from the generator that generates products from a plurality of generation models. The evaluator evaluates the products excluded by the information processing device 10, and the generator generates a new target based on the evaluation.

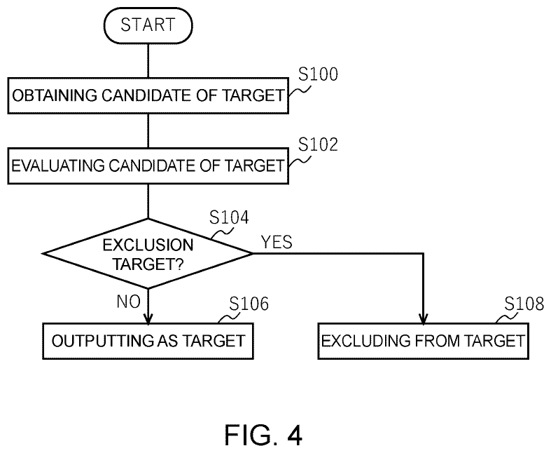

FIG. 4 is a flowchart showing a non-limiting example of processing of the information processing device 10 according to an embodiment. First, the information processing device 10 obtains data of a target as a candidate (S100). The data of the target may be generated by the information processing device 10 or may be generated by another information processing device.

In the case of crystal search, the data of the target as a candidate may be data relating to one or more crystal structures.

The information processing device 10 evaluates the obtained data of the target (S102). The evaluation model can be used for the evaluation. The evaluation model may be, for example, a model that obtains a feature quantity of the target when data relating to the target is input.

When performing crystal search, the evaluation model may be a model used in a Neural Network Potential (NNP) method. That is, the evaluation model may be a model that outputs a physical property value when a crystal structure is input. The physical property value may be, as a non-limiting example, the physical property value such as energy or force.

The information processing device 10 determines whether the candidate of the target is an exclusion target based on the obtained feature quantity (S104). When the information processing device 10 determines that the candidate is not the exclusion target (S104: NO), the information processing device 10 outputs the candidate as an evaluation target (S106).

When it is determined that the target is the exclusion target (S104: YES), the information processing device 10 extracts the candidate as the target to be excluded so that the target is not used in subsequent generation (S108).

In the processing of S106, the information processing device 10 can execute various processing depending on the configuration of the information processing device 10. For example, when the information processing device 10 is used only for determination to extract exclusions, the information processing device 10 can output candidates not determined as exclusion targets to the evaluator.

The information processing device 10, for example, executes the extraction of the exclusion targets using the evaluation model, and may execute the processing using the evaluation model as a part of the evaluator. That is, the information processing device 10 may calculate the evaluation value as an evaluator and execute determination of exclusion targets based on the evaluation value. In this case, the information processing device 10 can reflect candidates not regarded as exclusion targets in next generation by the generator, and can also exclude candidates regarded as exclusion targets so as not to affect the next generation by the generator.

The processing of S102 to S104 will be described more specifically.

The information processing device 10 can execute exclusion determination of target candidates using one or more evaluation models. As a non-limiting example, the information processing device 10 can determine whether each candidate target is the exclusion target by performing ensemble processing with a plurality of evaluation models.

The plurality of evaluation models can use different versions or different revisions of the same model. Further, in the plurality of evaluation models, at least one may be trained by a method different from other models. Further, in the plurality of evaluation models, at least one may be trained using training data different from other models. Further, in the plurality of evaluation models, at least one may be formed by a neural network model having a configuration different from other models.

Further, the plurality of evaluation models may be models with different parameters, models with different hyperparameters, models formed in different formats, or models trained using different training data, etc. Further, by acquiring the evaluation values under different conditions using the same evaluation model, it is also possible to substitute for the evaluation using the plurality of evaluation models.

Further, when one evaluation model is capable of switching modes, the information processing device 10 may acquire a plurality of feature quantities by switching modes in the one evaluation model and inputting candidates. In other words, one trained model serving as an evaluation model can output outputs corresponding to a plurality of modes as the evaluation values.

As described above, the plurality of evaluation models may be, as a non-limiting example, a plurality of models with different learning methods, a plurality of models with different network configurations, or a plurality of models with different operation modes. Although these are apparently one model, they can also be realized using a model capable of generating a plurality of kinds of outputs.

By using one or more evaluation models including models that output at least different feature quantities in this way, it becomes possible to realize the search that is robust over a wide range and has low cost.

As a non-limiting example, the information processing device 10 can also acquire the feature quantities using one or more evaluation models provided using an Application Programming Interface (API), and determine the exclusion targets based on each of the plurality of feature quantities acquired from the one or more evaluation models.

The information processing device 10 may, for example, acquire a predetermined feature quantity from the plurality of evaluation models, calculate an average value of the acquired plurality of predetermined feature quantities, and compare the average value with a threshold value to determine the exclusion target. Specifically, the information processing device 10 can acquire, as the evaluation values, the feature quantities output from the plurality of evaluation models for the feature quantity that is a value of approximately 0 in a normal system, and determine whether an absolute value of the average of the plurality of the evaluation values is equal to or less than the threshold value. When the absolute value of the average is equal to or less than the threshold value, it may be determined that the uncertainty is low (the certainty is high), and when it is higher than the threshold value, it may be determined that the uncertainty is high (the certainty is low).

As another example, the information processing device 10 may determine the exclusion target by comparing a plurality of predetermined feature quantities with a corresponding threshold value, respectively, and determining whether a predetermined number or more of results exceed (or below) the threshold value.

The information processing device 10 may, for example, acquire the predetermined feature quantity from the plurality of evaluation models, calculate a standard deviation of the acquired plurality of predetermined feature quantities, and determine that when the standard deviation is within a predetermined range, it is not excluded (the uncertainty is low or the certainty is high), and when it is outside the predetermined range, it is excluded (the uncertainty is high or the certainty is low).

Further, when acquiring the predetermined feature quantity by ensemble, the information processing device 10 can also perform weighting associated with each of the evaluation models. The information processing device 10 may, for example, acquire an output result from an ensemble model with weighting such that a model trained earlier has smaller weighting, or a model trained using a wider range of training data than others has larger (or smaller) weighting. As an example, the weighted average of the plurality of evaluation values may be calculated.

Further, the information processing device 10 can also output, for example, a result obtained by ensemble of the predetermined feature quantity as a weight in the next generation operation. In this case, the generator may use the weight not for determining whether to use/not use the candidate in the next generation operation, but for some processing based on the weight, for example, the probability of being selected in a genetic algorithm, the probability of being incorporated into crossover, the probability of being mutated, etc.

Further, instead of simply outputting a result regarding exclusion/non-exclusion, the information processing device 10 may perform processing to calculate a probability of exclusion in the next generation operation. The information processing device 10 may, for example, calculate the exclusion probability in the next operation for the candidate of interest based on results of the plurality of evaluation models. The information processing device 10 may output whether to exclude or not to the generator based on the exclusion probability, or as another example, the information processing device 10 may output the exclusion probability to the generator, and the generator may execute generation based on the exclusion probability.

The information processing device 10 may acquire two or more predetermined feature quantities from each of the evaluation models. The information processing device 10 may, for example, acquire a plurality of first feature quantities and a plurality of second feature quantities of the candidate targets from the plurality of evaluation models capable of acquiring the first feature quantity and the second feature quantity for the targets, and extract the exclusion targets based on these feature quantities. For each of the feature quantities, the result of ensemble of the plurality of evaluation models can be used.

Each of the examples described above can be used in combination within a non-contradictory range.

In summary, the information processing device 10 acquires the plurality of one or more predetermined feature quantities for one target from the one or more evaluation models, and calculates the uncertainty regarding whether to use as the data based on the acquired plurality of predetermined feature quantities. The information processing device 10 can determine whether to use the target in the next generation iteration based on the uncertainty.

The uncertainty is an index used for the above-mentioned determination of the exclusion. The uncertainty may be an index representing a degree to which the evaluation accuracy for the product deteriorates. In the case of CSP, the uncertainty can be, as a non-limiting example, a degree to which accuracy of NNP for the crystal structure deteriorates. The uncertainty can include, as a non-limiting example, model error included in an evaluation function in optimization and/or approximation error in formulation.

When used for crystal search, as described above, each of the plurality of evaluation models may be a model in the form of NNP. The information processing device 10 obtains the physical property values, for example, energy, for a certain crystal structure output from each NNP. Then, the information processing device 10 can calculate the uncertainty of the output value of the model for calculating the evaluation value relating to the crystal structure, that is, the evaluation model for the crystal structure, from the obtained plurality of energy values, and determine whether or not to perform the generation based on the crystal structure based on the uncertainty.

At the timing, the information processing device 10 can also use an energy value as the evaluation value. The evaluation value may be obtained from one predetermined evaluation model, or may be an energy value obtained as a result of ensemble of the plurality of evaluation models.

The information processing device 10 can also use the physical property value different from energy, for example, force or the like, as the physical property value. The information processing device 10 can also set energy and force as predetermined feature quantities. In this case, the information processing device 10 can also determine the uncertainty based on a plurality of energies and a plurality of forces obtained from the plurality of evaluation models.

The information processing device 10 may, for example, comprehensively consider information on energy (first predetermined feature quantity) and force (second predetermined feature quantity) output from NNP which is the evaluation model for the crystal structure, and calculate the uncertainty. Specifically, the information processing device 10 can set the threshold values for energy and force respectively, execute determination processing using the threshold values for each of the plurality of obtained feature quantities, and when the uncertainty of either one is high (the certainty is low), exclude the crystal structure or the like from the search target. Further, as a non-limiting example, when it is desirable to use information on a wider range of the targets such as at the initial stage of search, when the uncertainty of both is high (the certainty of both is low), the crystal structure or the like can be excluded from the search target.

In the case of CSP, the information processing device 10 may obtain the uncertainty using the obtained energy as the evaluation value, may obtain the uncertainty using the obtained force as the evaluation value, or may obtain the uncertainty using both the obtained energy and the obtained force as the evaluation values.

Based on the result, the generator may generate the crystal structures so as not to use, for example, the crystal structures excluded in the generation model, that is, so as not to generate the crystal structures in the vicinity of the excluded crystal structures. When using a genetic algorithm, for example, the generator may determine a parent generation so that the excluded crystal structure is not selected. Further, as another example, when using a genetic algorithm, the generator may perform processing of selection, processing of crossover, and processing of mutation with a probability based on the uncertainty output as described above.

The evaluation model may be formed as a model that outputs an index relating to the above-mentioned uncertainty together with energy.

The evaluation model may be in an aspect capable of internal dropout. In this case, the information processing device 10 can also obtain the plurality of physical property values by changing nodes to be deleted in dropout.

As described above, according to the present embodiment, by obtaining the uncertainty based on the one or more evaluation models in various search algorithms or algorithms for obtaining the targets to be excluded in various iterative calculations, prediction accuracy of extraction of the exclusion targets can be improved.

For example, in CSP, as the number of elements used increases, calculation cost and time cost increase exponentially, and in this case, pruning of the crystal structures that do not need to be searched becomes extremely important in the cost reduction and the accuracy improvement. By using the processing in the present disclosure, it is possible to improve the prediction accuracy of pruning of the crystal structures, and thereby to reduce cost of the crystal structure and improve the accuracy. Further, application to bulk, molecules, surfaces, and nanomolecules is also possible.

As described above, according to the present embodiment, performance of a search algorithm (an example of a generation method) can be improved. Further, according to the present embodiment, the accuracy of the evaluation function that can be one of the causes of performance degradation can be improved.

The above-described embodiments can also be used for optimization of reaction path search. Further, as described above, it can also be used for the selection of the next parent generation after mutation in a genetic algorithm. It can also be used for the uncertainty evaluation of surrogate models. In addition, it can be applied to various algorithms that require similar exclusion processing and probability processing.

For example, embodiments in the present disclosure can also be applied to structural optimization of wings using a fluid surrogate model. When executing the structural optimization of wings, by obtaining the uncertainty at a timing of obtaining the evaluation values for the structure of the structurally optimized wing, performance of the search algorithm can be improved.

An example of executing CSP as one embodiment will be described more specifically. According to the above-described embodiment, for example, the information processing device 10 generates the crystal structures or the like for the executing search, which are one or more first products, and then executes calculation of the uncertainty for the one or more first products. This is because, in CSP, a convex hull is calculated from search results, and search is realized in the range based on the convex hull. The convex hull conceptually includes a set of only structures with the most stable energy for each composition of a substance.

When the reliability of NNP is low and, for example, the crystal structure or the like which is a first product returns the physical property value such as an abnormally low energy value, if such the first product is not rejected, the shape of the final convex hull changes significantly. When the shape of the convex hull changes, results of the analysis after using the results of CSP change, and therefore, by using the uncertainty, it is desirable to evaluate the uncertainty each time the first product is generated so that inappropriate results do not enter.

Further, a method of selecting structures to be included in calculation of the convex hull after performing search is also conceivable, but in this case, at a timing of sequentially determining structures to be searched, there is a problem that the second products generated based on undesirable structures also have the undesirable structures.

Further, in order to effectively obtain the above-mentioned uncertainty in CSP, a configuration that reads the evaluations by the plurality of evaluators such as NNP with less overhead is generally required. Therefore, when the information processing device that executes CSP and the information processing device that executes the inference of NNP are implemented in the same information processing device, it is necessary to take measures such as reducing the size of the structure of “the first product” and “the second product that may be generated from the first product in the next iteration”. On the other hand, if the configuration allows NNP to be called via API, it becomes possible to call a plurality of NNPs more flexibly.

Of course, the above can also be applied to other than CSP. As a non-limiting example, it can also be applied when repeating processing such as the generation of atomic structures, the evaluation of generated atomic structures with NNP, and the generation of new atomic structures based on the evaluation results until a criterion is satisfied.

Here, processing of CSP can be summarized as follows, for a non-limited example.

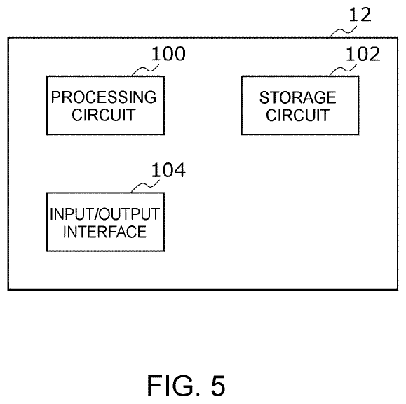

FIG. 5 is a block diagram schematically illustrating an information processing device 12 that executes CPS according to an embodiment. The information processing device 12 includes a processing circuit 100, a storage circuit 102, and an input/output I/F 104. The information processing device 12 searches for and outputs the structure of a compound based on the input information.

The processing circuit 100 is a circuit (processor) that realizes processing in the information processing device 12. The processing circuit 100 executes various processing based on the input information and/or data stored in the storage circuit 102.

The storage circuit 102 stores data necessary for processing of the processing circuit 100 and/or data necessary for starting the information processing device 12.

The input/output I/F 104 is an interface that connects the exterior and the interior of the information processing device 12. A user interface may be provided as a part of the input/output I/F 104.

In addition to the above, the information processing device 12 can be appropriately provided with configurations necessary for the information processing device 12 to execute processing, such as a power supply unit and a control unit.

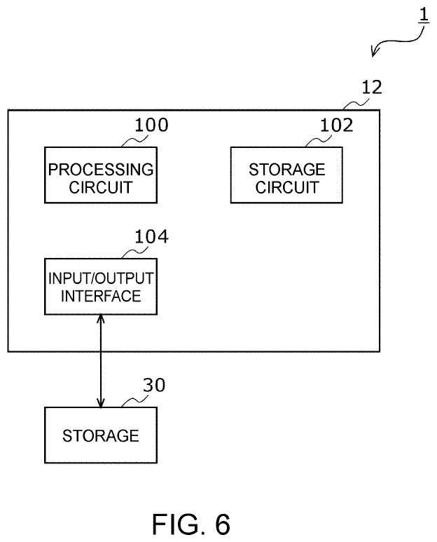

FIG. 6 is a diagram schematically illustrating an example of an information processing system 1 according to an embodiment. The information processing system 1 includes the information processing device 12 and a storage 30.

The storage 30 can store at least a part of data necessary for processing of the processing circuit 100. The processing circuit 100 can obtain data stored in the storage 30 via the input/output I/F 104 and execute processing. The storage 30 may be, for example, a configuration for storing data such as a file server or a part of a server provided on a cloud.

As shown in this figure, instead of the storage circuit 102 storing all data necessary for processing of the processing circuit 100 inside the information processing device 12, data processed by the processing circuit 100 and/or data intermediately obtained in the processing of the processing circuit 100 can be stored in the storage 30 provided outside the information processing device 12. Thus, the information processing system 1 can have a configuration in which a part of the function of the storage circuit 102 is outside the information processing device 12.

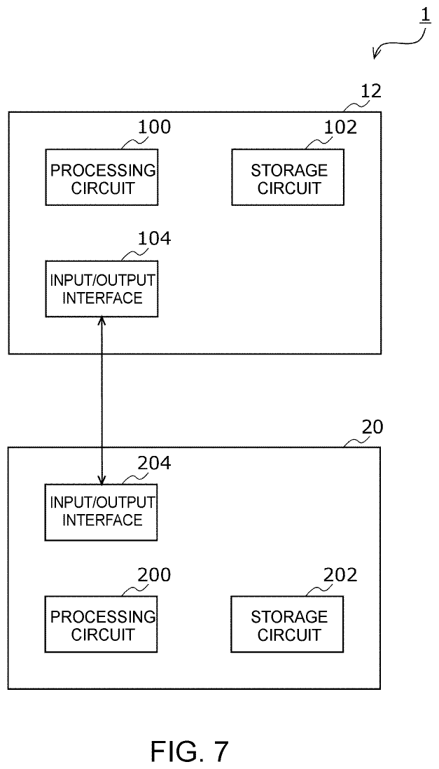

FIG. 7 is a diagram schematically illustrating an example of an information processing system according to an embodiment. The information processing system 1 includes at least the information processing device 12 and an information processing device 20.

The information processing device 12 is, for example, an information processing device that operates as a server, and the information processing device 20 is, for example, an information processing device that operates as a client.

The information processing device 20 includes a processing circuit 200, a storage circuit 202, and an input/output I/F 204. The information processing device 20 makes a request for searching a structure of a compound to the information processing device 12 together with information. The information processing device 12 executes a search for the structure of the compound based on the information and the request obtained from the information processing device 20.

The processing circuit 200 is a circuit (processor) that executes processing in the information processing device 20. The processing circuit 200, for example, receives a request from a user via the input/output I/F 204, and transmits the request to the information processing device 12 via the input/output I/F 204.

The storage circuit 202 stores data necessary for operation of the information processing device 20. Further, the storage circuit 202 can also store calculation results of the information processing device 12 obtained via the input/output I/F 204.

The input/output I/F 204 is an interface that connects the exterior and the interior of the information processing device 20.

The input/output I/F 104 and the input/output I/F 204 can be connected via, for example, a wired network or a wireless network. In this case, the input/output I/F 104 and the input/output I/F 204 include an appropriate network I/F.

In addition to the above, the information processing system 1 may include the storage 30 in FIG. 2, or may include one or more information processing devices 12 for one or more information processing devices 20. When the plurality of information processing devices 12 are provided, a plurality of processors may cooperate to process the requests from the one or more information processing devices 20.

In this form, the information processing device 12 may be an information processing device provided on a cloud, or may be a device that provides services to the information processing device 20 in an aspect of Software as a Service (SaaS).

A stable structure has lower energy than neighboring structures. When a plurality of elements are mixed, if the structure has lower energy, the compound can be said to have a stable (crystal) structure. By referring to a graph showing a state, it is possible to know what composition ratio is the stable structure. For example, the structure on the convex hull can be said to be the stable structure.

In the present disclosure, the structure search of the compound is performed by updating the convex hull. The structure search is to search for the structure of the substance having a structure that is more energetically stable or a structure having a desired physical property value.

In the present disclosure, a substance is various materials, etc., and is a concept including compounds or simple substances. For example, the substance may be a simple substance crystal, a carbon nanotube, a fullerene, a nanoparticle, or the like. As structure search of the substance, for example, the structure of a surface of a substance, a molecular structure, or the like may be searched. In the following, when simply described as a structure, it may be read as an atomic structure including an atomic arrangement, an atomic array, or the like of the substance.

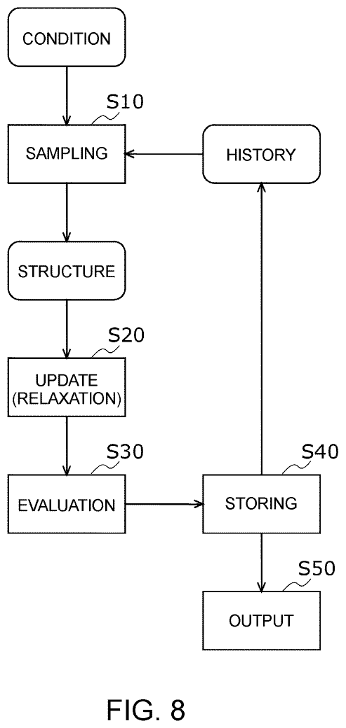

FIG. 8 is a diagram schematically showing a flow of structure search processing for substances by the information processing device 12. In the following, processing may be described as processing of the information processing device 12 or the information processing device 20, but this can be read as processing of the processing circuit 100 or the processing circuit 200, respectively, within a non-contradictory range.

The information processing device 12 samples the structure based on the given conditions (S10).

The information processing device 12 updates (relaxes) the sampled structure (S20).

The information processing device 12 evaluates the updated structure (S30).

The information processing device 12 stores the structure as a history together with the evaluation (S40).

In the second and subsequent iterations (S10 to S40), the information processing device 12 performs sampling based on the evaluation from the history. Further, when a predetermined calculation completion condition is satisfied, the information processing device 12 outputs the structure according to the evaluation value (S50).

Each processing in the above processing will be described in more detail.

### (Initialization of History)

When a search using the same combination of elements has been executed in the past, the information processing device 12 may use, as the history, information relating to results of the past search or intermediate progress.

When executing the search using the combination of the elements that has never been executed before, or when executing a new search that does not rely on the past results for the combination that has been executed before, the information processing device 12 can set a structure in which elements are randomly arranged as an initial structure. For example, the information processing device 12 can use, as an initial history, a plurality of structures in which elements obtained as the conditions are randomly arranged in a cell in Density Functional Theory (DFT) calculation.

As a non-limiting example, the information processing device 12 can also arrange elements by dividing a cell into a plurality of subcells, randomly arranging elements in one subcell, and copying the subcell to fill the cell.

### (S10: Sampling of Structure)

The information processing device 12 can use, as a non-limiting example, an evolutionary algorithm in the sampling of the structures. The information processing device 12 samples the structures using, for example, a genetic algorithm.

A genetic algorithm, which is one of the evolutionary algorithms, selects the samples from the plurality, generally a huge amount, of samples that are the candidates for a parent generation to form the parent generation. In samples belonging to the parent generation, crossover between samples and mutation within samples are performed to generate the candidates for the samples of a child generation.

In the present disclosure, the term “sampling of the structures” or simply “sampling” indicates “processing of selection of the samples of the parent generation” and “processing of the sample generation of the child generation through processing including at least crossover (mutation may also be performed)”.

The information processing device 12 can perform the selection using, for example, energy values for the respective structures (samples) obtained as the evaluations in the previous iteration. For example, when it is desired to obtain the crystal structure composed of two elements, the composition on a two-dimensional convex hull can be selected as an elite sample, with the ratio of the two elements on the horizontal axis and energy [eV/atom] in the structures at each of various arrangements and the like at the element ratio on the vertical axis.

The information processing device 10 according to the embodiment described above determines whether to accept the structure by obtaining the uncertainty relating to each structure.

In addition to this, the information processing device 12 can also perform the selection based on a multi-objective optimization method using indices relating to stability or desired physical characteristics such as the plurality of physical property values, not only energy. In this case, the information processing device 12 can also execute the selection using the other evaluation values obtained in the evaluation processing of S30 described later, as a non-limiting example.

As a non-limiting example, the information processing device 12 can perform the elite selection based on a semi-ordered sequence using a predetermined index as a sort key. Semi-order is generally a relation in which reflexivity, antisymmetry, and transitivity are established.

In one embodiment, p<=q for structures p and q means that when E(p)<=E(q) and Comp(p)=Comp(q), “<=” satisfies the semi-order law. Here, E(p) indicates the energy of structure p or the value of the index for sorting, and Comp(p) indicates the composition ratio of p (for example, the composition ratio of two elements). That is, in other words, for different samples p and q having the same composition ratio, when the index value indicates E(p)<=E(q), it is described that p<=q.

The information processing device 12 calculates the value E serving as an index for the each structure of the samples stored in the history. The information processing device 12 executes non-dominated sorting on the E, and extracts samples in order from those with smaller ranks. The information processing device 12, for example, when there are a plurality of structures having the same element composition ratio, sets ranks for these structures in order of smaller indices, and selects samples in order from those with smaller ranks.

Further, the information processing device 12 can select samples in order of smaller ranks, and at ranks exceeding the number of the samples to be selected, select the samples using crowding distance D. The crowding distance D of sample p can be, for example,
D(p)=(Manhattan distance from two samples in the vicinity of p), or
D(p)=(average of Manhattan distances from a plurality of samples in the vicinity of p), or
D(p)=(Manhattan distance from a plurality of samples in the vicinity of p)+(normalized energy gap of p), or the like.
   The vicinity indicates other samples that are close in terms of indices, for example, energy values.

By performing the selection, the information processing device 12 can execute the elite selection, but the samples selected in this way are always vertices of the convex hull in a multidimensional space (for example, a space formed by energy values for composition ratios of a plurality of elements). For this reason, the selected samples are highly likely to be the samples showing the structures that have not been improved for long iterations.

The information processing device 12 can filter the samples before the non-dominated sorting in order to appropriately select or not select such samples. Filtering can be expressed, for example, by the following equation.

$$normalized ( E ( t ) ) \times {α}^{g ( t ) - {g}^{*} ( t )} \leq ε \qquad ( 1 )$$

$$normalized ( E ( t ) ) = \frac{{E}_{max} - E ( t )}{{E}_{max} - {E}_{min}} \qquad ( 2 )$$

The g(t) is the current generation of t, and the g*(t) is a generation of the t that satisfies the following.

argmin{E(s)|s∈history,|r(t)−r(s)|≤δ} (3)

The history represents the entire structure (history) that has been searched so far. The r(t) is a multidimensional vector representing the composition of the structure t, and α, ε, and δ are hyperparameters. Further, |⋅| indicates the Manhattan norm of the multidimensional vector. The information processing device 12, for example, excludes the t satisfying equation (1) from the candidates for the elite selection.

By executing non-dominated sorting and crowding distance sorting on the candidates selected in this way, the information processing device 12 can execute the elite selection. According to the selection, it is possible to exclude individuals belonging to a region where a large number of structures exist in the vicinity, that is, a region where a large number of individuals having similar genes have already been evaluated, and to preferentially select individuals in other regions, that is, regions where not much search has been executed in the history, as samples for the next generation.

After selecting samples, the information processing device 12 generates samples of a child generation by using the selected samples themselves, crossover of a plurality of selected samples, and mutation of the selected samples.

When using the selected samples themselves, the information processing device 12 uses the structure in the history of the parent generation as it is as one of the samples of the structure of the child generation. For example, one of the samples selected as an elite is set as a structure belonging to the child generation.

The crossover can be executed, for example, in units of cells in DFT calculation or the like. By replacing the structure of elements in at least a part of a region in the structure indicated in the cell in a certain sample with a structure of elements in a part of a region in another sample, a crossed sample can be obtained.

Further, the crossover is not limited to the above, and may be executed, for example, by exchanging a part of the structure represented by a graph with a part of another graph structure, or by exchanging a part of the structure described by SMILES with a part of another structure described by SMILES.

When mutating the sample, the information processing device 12 changes at least a part of types of elements, arrangement of at least a part of elements, and/or connection between at least a part of elements in the structure in the sample to another one. For example, the structure of a sample can be mutated to be used as the sample.

The information processing device 12 can, for example, swap two different types of elements in the structure in one sample to cause the mutation. The information processing device 12 can, for example, move positions of four elements having a rectangular shape in one sample so as to become a parallelogram to cause mutation. As some other examples, the information processing device 12 can cause the mutation by arranging (adding) or deleting a random element at a random position, or randomly changing a type of element.

In addition, both the crossover and the mutation for a certain two samples may be executed to generate one sample of the child generation. For example, the sample of the child generation may be generated from a certain sample and another sample.

In this way, the information processing device 12 executes the sampling of the structure of a substance by a genetic algorithm. Of course, the information processing device 12 can similarly execute sampling by the method of another evolutionary algorithm.

Further, the information processing device 12 can also execute sampling by a method based on a surrogate model such as Bayesian optimization, a sampling-based method such as Markov chain Monte Carlo method (MCMC), or a method based on symmetry of space groups.

### (S20: Update (Relaxation), S30: Evaluation)

After the sampling, the information processing device 12 updates the samples so that the structure is established as a stable structure or the structure having a desirable physical property value for each of the sampled structure.

The structure obtained by executing the above-described the crossover and the mutation may not be stable as a crystal structure, for example, the distance between atoms may be too close. In order to update the generated unstable structure to a stable structure, the information processing device 12 executes relaxation processing.

The information processing device 12 can, for example, locally optimize the sampled structure using NNP.

The information processing device 12 calculates energy by applying NNP to the sampled structure. The information processing device 12 executes the optimization of the structure so as to have a locally stable structure so that the energy value becomes smaller. The information processing device 12 can, for example, execute the local optimization of the structure by obtaining a position derivative with respect to energy.

By the processing, the information processing device 12 updates (relaxes) the sample obtained by the crossover and/or the mutation with a stable structure in the vicinity of the sample. In other words, the information processing device 12 updates (relaxes) the sample to a more stable structure.

More specifically, the information processing device 12 updates the sample to a sample indicating a point where a gradient of the potential of NNP becomes 0 by applying a Broyden-Fletcher-Goldfarb-Shanno (BFGS) Algorithm or the like using the target sample as an initial value. The vicinity may indicate some region including such a point where the gradient becomes 0 and the sample (before update). What shape such a region has depends on the shape of the potential surface of NNP and the position of the sample.

The information processing device 12 can adopt any appropriate method as an optimization method. The information processing device 12 can, for example, locally adopt the BFGS method and obtain the stable structure from energy values of the structures in the vicinity. In addition, the information processing device 12 can relax the structure using a hill climbing method, a quasi-Newton method, or the like, and update the sampled structure.

By the processing, it becomes possible to update a structure that is highly likely to be inappropriate in the structure obtained from the genetic algorithm to a more stable structure and execute a search.

Note that, in the processing of S20, the information processing device 12 can also perform the evaluation of the sampled structure of S30. That is, the evaluation value obtained for optimization in the processing of S20 can also be used as the evaluation value in S30.

In the processing of S10, when an index different from the evaluation used in the local optimization of S20 is used as an evaluation, processing for calculating the index can be performed on the sample updated after completion of the processing of S20. Even in this case, when the evaluation value used in the processing of S20 is part of the evaluation in S30, the evaluation value obtained in the progress of S20 can be used.

The information processing device 12 can, for example, calculate energy of each of the relaxed structures, and evaluate the sampled structure by obtaining a difference between the calculated energy and energy with the true convex hull.

Since search is generally executed in a situation where the true convex hull is unknown, instead of this, the information processing device 12 can calculate and evaluate a distance between the current convex hull of energy and the energy of each calculated structure, for example, a distance with a structure belonging to the current convex hull.

Further, as another example, the information processing device 12 can also use, as the convex hull, the energy value that is known, for example, published as a database, and use the distance between the convex hull and the convex hull of the sampled structure as the evaluation.

Further, as another example, the information processing device 12 can use an area or line segment length of the convex hull as an overall evaluation, or can use the distance between a point indicating a certain configuration and the convex hull as the evaluation.

### (S40: Storage, S50: Output)

The information processing device 12 stores the evaluated result in association with the evaluated structure (S40). The stored structure is registered as the history and can be used as the sample of the parent generation in the next iteration.

The information processing device 12 repeats the processing from S10 to S40 until an end condition is reached. The end condition can be set similarly to general optimization, such as until the number of structures for which the evaluation has been appropriately completed reaches a predetermined number, until a time required for the search exceeds a predetermined time, until a predetermined physical property value of the searched structure reaches a predetermined value, or until a predetermined number of iterations are executed. After satisfying the end condition, the information processing device 12 outputs the information of samples finally obtained (S50).

The information processing device 12 can output, for example, structures corresponding to samples constituting the convex hull, or can also output structures whose distance from the convex hull is within a predetermined value, for example, within a value such as 0.05 [eV/atom]. In such configurations, errors occur in calculations of NNP, DFT, etc., and if the configuration is within the above-mentioned predetermined value, there is a possibility that it can be actually synthesized, and in such a case, it is meaningful to output as a candidate for search results. Further, when search is performed based on the physical property values, it is conceivable that stable or metastable structures in which the physical property value is in a desired range can be returned.

By performing optimization or output related to the convex hull that can be obtained for evaluation or sampling, the information processing device 12 can execute search based on a Pareto front in multi-objective optimization, or search based on optimization of a pseudo Pareto front, and output the results.

By using the processing of obtaining the uncertainty in the information processing device 10, it becomes possible to maintain the convex hull in a desirable shape.

The information processing device 12 can output, for example, as information relating to a structure, types of elements, positions of atoms, and a cell structure, which are parameters that determine the crystal structure. Further, the information processing device 12 can output energy or the physical property values.

The information processing device 12 may also output the search history. In this case, it becomes possible to present to a user information such as how search was executed, for example, whether efficient search is being executed.

Further, the information processing device 12 can provide a viewer, and output information for visualizing structures. The output can be presented, for example, via the input/output I/F 104, 204.

Note that the information processing device 12 can also appropriately output intermediate progress even during execution of search. The information processing device 12 can output not only structures, but also, for example, information on the evaluation values as a graph, or output a state of the convex hull as a graph.

According to the embodiment, the information processing device 12 can automatically search for a structure of a substance composed of the elements by, for example, simply inputting the conditions such as types of elements to be used. Therefore, for example, by the information processing system 1 shown in FIG. 7, it becomes possible to provide a service as SaaS in which high-speed calculation is executed on the server side by simply inputting information on types of elements (for example, a substance containing Li and In, or a crystal containing Ti and O) from the client side, and search results are output to the client.

Calculations based on classical theories, which can be the evaluation values such as energy, such as first-principles calculation, calculation by molecular dynamics method, or calculation by density functional theory, require a long time for calculation for each structure.

When using a genetic algorithm as a non-limiting example of sampling, the information processing device 12 executes this very long time multiple times to evaluate a plurality of samples of the child generation, and may require a time of several days to several weeks, for example, to obtain the evaluation values for one generation and obtain stable structures.

By using the NNP method, calculation of the evaluation value and relaxation processing of the structure can be realized at high speed and with high accuracy. As a result, according to one embodiment, it becomes possible to realize global and robust search for substance structures using a method of an evolutionary algorithm such as a high-speed genetic algorithm.

When using a genetic algorithm as an example of sampling, it becomes possible to simultaneously realize optimization for a plurality of samples. As a result of being able to increase the number that can be sampled, high-speed and global structure search can be realized. By using NNP, it becomes possible to realize evaluation, which has been a bottleneck in search using a plurality of samples simultaneously, at high speed. In one embodiment of the present disclosure, by using NNP in this way, it becomes possible to realize substance structure search at high speed while ensuring robustness.

By using a plurality of samples simultaneously, it becomes possible to realize processing with higher accuracy. Further, by speeding up evaluation by NNP, it becomes possible to expand the range of search more than before by a plurality of general-purpose accelerators such as GPUs, even without using a supercomputer.

Further, information that can be input as conditions is not limited to combinations of elements. For example, the information processing device 12 may accept information relating to the environment such as temperature and pressure as conditions. In this case, the information processing device 12 can reflect information on the environment such as the temperature and pressure in obtaining the physical property values in NNP, and can realize local search for stable structures under conditions with higher accuracy. As a result, the information processing device 12 can execute search results as a whole processing based on conditions more.

Information input as conditions can be input by a user, for example, via the information processing device 20 on the client side.

As conditions, a combination of elements and a composition ratio of these elements may be set. The composition ratio may be expressed as a range. In this case, the information processing device 12 can, as a non-limiting example, execute processing based on the composition ratio (for example, initial sample generation, crossover, mutation) at a timing of sampling. When the number of elements increases, it becomes possible to limit the search range.

The information processing device 12 can, for example, perform sampling by determining a range of the composition ratio of Li to In. By this sampling, it is possible to search for structures of substances in different states such as a charged state and a discharged state.

The information processing device 12 can, for example, perform sampling by determining a range of an existence ratio of a certain element between a plurality of layers constituting a semiconductor. By this sampling, it is possible to search for structures such as atomic arrangements between fixed semiconductor layers. Semiconductors may undergo large changes in the physical property values even when atoms to be converted are minute, but it is possible to execute search covering such changes in the physical property values.

The information processing device 12 can, as an example of a search range, specify a range in a curve formed by an already obtained convex hull and perform sampling. By this sampling, it is possible to execute search within the specified range with high granularity.

As conditions, a range of the physical property values such as energy or density may be set. In this case, the information processing device 12 can execute local optimization in the case of using NNP with higher accuracy. Further, in evaluation using multi-objective optimization, it also becomes possible to obtain structures closer to conditions.

As conditions, a possibility of synthesis for obtained structures may be output. The possibility of synthesis may be determined based on existing processes.

As conditions, it may be an embodiment in which an evaluation function (evaluator) to be used can be set.

The information processing device 12 can output new structures themselves as described above. In addition to this, the information processing device 12 can also output at least one of a state of a convex hull of energy or other variables used for evaluation, visualization of which parent each structure was obtained from (including time-series changes), optimization history, or the physical property values of structures (crystal groups, cell structures).

According to the above embodiments, since calculation of the physical property values serving as evaluation can be realized at high speed using NNP, the number of individuals in one generation of an evolutionary algorithm can be increased. In other words, it becomes possible to further increase parallelism when using accelerators with multiple cores, using clusters, or using grid computing. As a result, it becomes possible to improve calculation speed and accuracy by widening the search range.

When using the configuration of FIG. 7, it may be an embodiment in which the information processing device 12 executes search by an evolutionary algorithm and the information processing device 20 executes evaluation. Further, as another example, it may be an embodiment in which the information processing device 12 executes search by an evolutionary algorithm and another information processing device 12 executes evaluation. In this way, each processing can be appropriately executed in an appropriate information processing device.

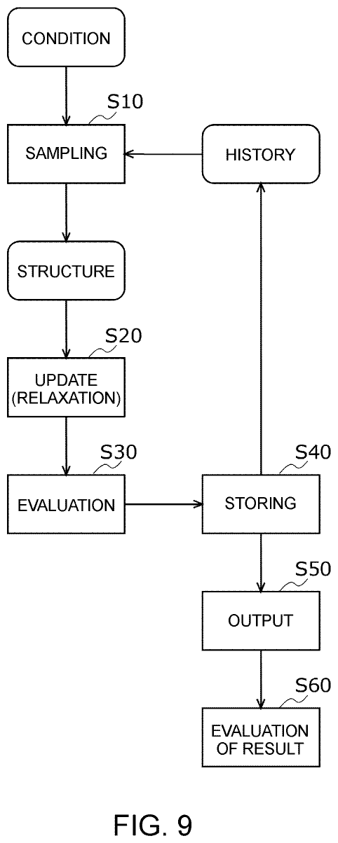

FIG. 9 is a diagram showing an aspect for evaluating results output in the above embodiment. The information processing device 12 in FIGS. 5 to 7, or the information processing device 20 in FIG. 7, can evaluate results for which search has been completed (S60).

The information processing device 12 or the information processing device 20 may, for example, set a structure of a sample output as a search result as a candidate for a structure to be obtained, and in this case, by evaluating a plurality of candidate structures obtained in the search, a most desirable structure can be extracted from the candidates.

For example, in addition to the evaluation in S30, the physical property values such as energy can be obtained by classical DFT calculation. By evaluating in this way, by calculating energy values or the like using another calculation method for candidates searched at high speed using NNP, accuracy of obtaining a desired structure can be increased.

Further, when a known convex hull is used as an evaluation target, the information processing device 12 or the information processing device 20 may calculate values such as energy for structures belonging to the known convex hull by a classical method or a method using NNP. By obtaining energy values or the like of known structures in this way, it also becomes possible to confirm whether evaluation in the information processing device 12 was appropriately executed.

The information processing device 12 obtains at least information relating to a search target from the information processing device 20.

The information relating to a search target represents information obtained from the information processing device 20 used by a user. The information relating to a search target can include information such as types of elements constituting a substance and composition ratios described in the embodiments. Without being limited to these, the information can include some information indicating a substance to be a search target or some information indicating search conditions (temperature, pressure, range of convex hull, range of the physical property values, etc.). Further, the information processing device 12 may obtain information relating to a search target from the information processing device 20 via another device.

The information processing device 12 obtains structures of a plurality of substances based on the obtained information relating to a search target, and updates the obtained structures of a plurality of substances.

Obtaining structures of a plurality of substances may be a concept including at least one of the following.

That is, the information processing device 12 may obtain structures of a plurality of substances by sampling.

Further, the information processing device 12 may obtain structures of a plurality of substances by random arrangement.

Further, the information processing device 12 may obtain structures of a plurality of substances based on history information. Even when performing elite selection using history, the information processing device 12 may set at least one of candidates to be selected as a structure of a substance generated by random arrangement. By doing so, global search can also be incorporated into local search.

Further, information on a structure of a substance may be a concept including at least one of the following.

That is, information on a structure of a substance may be information including types of atoms (elements) and position information of atoms (elements).

Further, information on a structure of a substance may be information including information on a cell structure.

Note that the information processing device 12 may have a configuration in which at least one structure of a substance is obtained based on the obtained information relating to a search target, and the obtained at least one structure of a substance is updated.

The information processing device 12 can evaluate updated structures of a plurality of substances using a neural network (for example, NNP), and transmit information on a searched structure of a substance to the information processing device 20 based on evaluation results. Further, the information processing device 12 may transmit information on a searched structure of a substance to the information processing device 20 via another device.

NNP may be considered to be a type of neural network.

This does not exclude the use of other neural network models in addition to neural networks that construct graphs based on so-called atomic arrangements, and evaluation of structures by these other neural network models. That is, NNP in the present disclosure does not exclude the use of neural network models for obtaining information relating to structures of substances as a broader concept.

The information processing device 12 can update obtained structures of a plurality of substances using a neural network.

The neural network may be the neural network of the above-mentioned NNP, or may be a different model. Note that the information processing device 12 may have a configuration in which information on updated at least one structure of a substance is evaluated using a neural network.

Further, the information processing device 12 may have a configuration in which information on one or more searched structures of substances is transmitted to the information processing device 20.

As described above, the information processing device 20 may be a plurality of devices.

Similarly, the information processing device 12 may be configured as a system including a plurality of devices.

The information processing system 1 described in the present disclosure may include one or more information processing devices. Similarly, the information processing system 1 can further include one or more storages 30.

In the information processing system 1, these configurations are typically connected by network lines using wireless means or wired means, such as the Internet, but connection methods are not limited to these, and means capable of appropriately connecting these configurations can be used.

As an example of the configuration, the information processing system 1 can also provide SaaS with the information processing device 12 as a server and the information processing device 20 as a client.

Some or all of each device (such as the information processing device 10, etc.) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

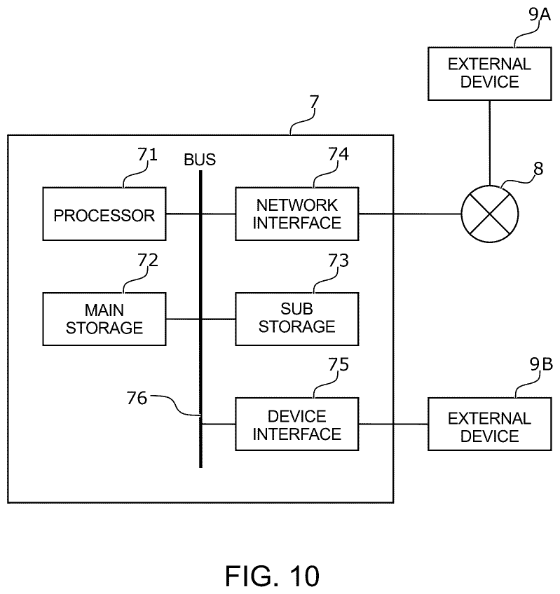

FIG. 10 is a block diagram illustrating an example of a hardware configuration of each device (such as the information processing device 10, etc.) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 10 is provided with each component one by one but may be provided with a plurality of the same components. Although one computer 7 is illustrated in FIG. 10, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (such as the information processing device 10, etc.) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (such as the information processing device 10, etc.) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (such as the information processing device 10, etc.) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (such as the information processing device 10, etc.) in the above embodiments may be enabled by the main storage device 72 or the auxiliary storage device 73 or may be implemented by a built-in memory built into the processor 71. For example, the storages 102 in the above embodiments may be implemented in the main storage device 72 or the auxiliary storage device 73.

In the case of each device (such as the information processing device 10, etc.) in the above embodiments is configured by at least one storage device (memory) and at least one processor connected/coupled to/with this at least one storage device, the at least processor may be connected to a single storage device. Or the at least storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (such as the information processing device 10, etc.) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrical connection/coupling,” “communicative connection/coupling,” “operative connection/coupling,” “physical connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

An embodiment of the present disclosure is expressed as a non-limiting example, as follows:

(1) An information processing device comprising at least one memory; and at least one processor configured to obtain one or more first products; evaluate uncertainty of evaluation values for each of the one or more first products using information from one or more evaluators; and obtain one or more second products based on an evaluation result of the uncertainty.

In the embodiment, the one or more evaluators may be one or more evaluation devices.

The evaluator may be implemented by using an evaluation model. The evaluator may be, for example, NNP. The evaluator may be formed with other neural network models, for example. The evaluator may be a model that outputs an index relating to uncertainty. In this case, outputting an evaluation value itself is not an essential configuration. That is, it is also possible to convert outputs from various models by a function or the like into the evaluation values. The evaluator may be configured with, for example, a trained machine learning model. Further, the evaluator may be a predetermined function or a model having mathematical meaning. The evaluator may be the evaluation device including an evaluation model.

The evaluator may have a configuration including, as a measurement device, a device that measures the evaluation values.

The evaluator may be a configuration included in the above-mentioned information processing device, or may be configured with one or more other information processing devices. The one or more other information processing devices may be connected to the above-mentioned information processing device by any connection capable of appropriately realizing data transfer.

Information from the one or more evaluators may be, as a non-limiting example, a value indicating uncertainty instead of the evaluation value.

As some non-limiting examples, the evaluation value may be an index representing a feature amount of a product, a physical property value of a product, or characteristics/properties/nature of a product.

As some non-limiting examples, uncertainty may include an index indicating whether or not accuracy of the evaluation value is high, may include information indicating whether or not to use for generation of a second product, or may include information indicating whether or not evaluation results using one or more evaluation models for a first product are reliable. The above-mentioned information or numerical values may be expressed as continuous values or may be expressed as discrete values.

As some non-limiting examples, obtaining the one or more second products (crystal structures, etc.) may include selecting the one or more second products from the one or more first products, or may include generating the one or more second products based on the one or more first products. Here, “based on generation results of the first products” can include being based on the first products themselves, being based on the generation results of the first products, or, for example, generating products in the vicinity of the first products.

As a non-limiting example, the evaluation of uncertainty includes evaluating certainty. Further, similarly, being based on the evaluation results of the uncertainty includes being based on the evaluation results of the certainty. That is, the above expression includes evaluating certainty of the first product and generating the second product based on the evaluation results of the certainty.

(2) The information processing device according to (1), wherein the at least one processor determines validity of each of the one or more first products based on the evaluation result of the uncertainty; selects the one or more first products determined to be valid as one or more valid products; and determines the one or more valid products to be the one or more second products.

As some non-limiting examples, the validity may include determination of whether or not the uncertainty is lower (or higher) than a predetermined criterion, or may include determination of whether the evaluation value obtained from the first product, or to be obtained from the first product, reflects reality.

(3) The information processing device according to (1) or (2), wherein the at least one processor selects, among the one or more first products, the first product to be used for generation of the one or more second products, based on evaluation results of the uncertainty, and generates the one or more second products using the selected first product.

As a non-limiting example, the evaluation results of the validity can be used instead of the evaluation results of the uncertainty.

As some non-limiting examples, this can include selection of the first products or exclusion of the first products not to be used. For example, in the context of CSP, this can include excluding from information for generating the next generation, and/or excluding from consideration of the final results.

(4) The information processing device according to any one of (1) to (3), wherein the at least one processor evaluates the uncertainty using a plurality of the evaluation values output by the one or more evaluators.

(5) The information processing device according to (4), wherein the at least one processor inputs the one or more first products to the one or more evaluators to obtain a plurality of the evaluation values, and selects the first product to be used for obtaining the one or more second products based on the obtained plurality of the evaluation values and a predetermined value.

As some non-limiting examples, the predetermined value may be a threshold value, may be a numerical value to be compared with a numerical value calculated based on a plurality of evaluation values, or may be a value representing a predetermined criterion.

(6) The information processing device according to (5), wherein the at least one processor obtains a probability of excluding a first product based on the plurality of the evaluation values, and determines the first product based on the probability.

(7) The information processing device according to (5), wherein the at least one processor selects the first product to be used for obtaining the one or more second products based on weights based on the one or more evaluators and the obtained plurality of the evaluation values.

(8) The information processing device according to any one of (1) to (7), wherein each of the one or more evaluators is a machine learning model.

(9) The information processing device according to (8), wherein the machine learning model is implemented in another information processing device.

(10) The information processing device according to any one of (1) to (9), wherein the at least one processor repeatedly executes obtaining of the one or more first products, the evaluating of the uncertainty, and the obtaining of the one or more second products.

As a non-limiting example, in the second and subsequent repetitions, the second products may be obtained as the first products. That is, obtaining the one or more first products may be the same operation as obtaining the one or more second products.

(11) The information processing device according to any one of (1) to (10), wherein each of the one or more first products is a crystal structure.

(12) The information processing device according to (11), wherein the at least one processor executes Crystal Structure Prediction (CSP).

(13) The information processing device according to (11), wherein each of the one or more evaluators is a model using a Neural Network Potential (NNP), and the evaluation value indicates at least one of energy or force of the first product.

(14) The information processing device according to any one of (11) to (13), which provides services in a form of Software as a Service (SaaS).

(15) The information processing device according to any one of (1) to (14), wherein the at least one processor receives a request from another information processing device; and stars, after receiving the request, execution of the obtaining of the one or more first products, the evaluating of the uncertainty, and the obtaining of the one or more second products.

(16) An information processing system comprising at least one first information processing device configured to obtain one or more first products, evaluate uncertainty of an evaluation value for each of the one or more first products using information from one or more evaluators, and obtain one or more second products based on an evaluation result of the uncertainty; and at least one second information processing device configured to receive a calculation request from a user, and send an execution request to the at least one first information processing device in accordance with the calculation request, wherein the at least one first information processing device starts the execution of the obtaining of the one or more first products, the evaluating of the uncertainty, and the obtaining of the one or more second products, after receiving the execution request from the at least one second information processing device.

(17) An information processing method comprising obtaining, by at least one processor, one or more first products; evaluating uncertainty of an evaluation value for each of the one or more first products using information from one or more evaluators; and obtaining one or more second products based on evaluation results of the uncertainty.

This information processing method can include executing at least part of the processing of the information processing device exemplified above by at least one processor.

(18) The information processing method according to (17), wherein the obtaining the one or more second products comprises determining, by the at least one processor, validity of each of the one or more first products based on the evaluation result of the uncertainty; selecting the one or more first products determined to be valid as one or more valid products; and determining the one or more valid products to be the one or more second products.

(19) The information processing method according to (17) or (18), wherein the obtaining the one or more second products comprises: selecting, by the at least one processor, from among the one or more first products, the one or more first products to be used for generating the one or more second products based on the evaluation result of the uncertainty; and generating, by the at least one processor, the one or more second products using the selected first products

(20) The information processing method according to any one of (17) to (19), wherein the evaluating the uncertainty comprises evaluating, by the at least one processor, the uncertainty using the evaluation value output by more than one of the one or more evaluators.

(21) The information processing method according to (20) further comprising selecting, by the at least one processor, the one or more first products to be used for obtaining the one or more second products based on the evaluation value and a predetermined value.

(22) A program for causing at least one processor to execute information processing comprising obtaining one or more first products; evaluating uncertainty of an evaluation value for each of the one or more first products using information from one or more evaluators; and obtaining one or more second products based on evaluation results of the uncertainty.

This program can include a program for causing at least one processor to execute at least part of the processing of the information processing device exemplified above.

(23) A non-transitory computer readable medium storing a program that causes at least one processor to perform information processing comprising obtaining one or more first products; evaluating uncertainty of an evaluation value for each of the one or more first products using information from one or more evaluators; and obtaining one or more second products based on an evaluation result of the uncertainty.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
at least one memory; and
at least one processor configured to:
obtain one or more first products;
evaluate uncertainty of an evaluation value for each of the one or more first products using information from one or more evaluators; and
obtain one or more second products based on an evaluation result of the uncertainty.

2. The information processing device according to claim 1, wherein the at least one processor is configured to:
determine validity of each of the one or more first products based on the evaluation result of the uncertainty;
select the one or more first products determined to be valid as one or more valid products; and
determine the one or more valid products to be the one or more second products.

3. The information processing device according to claim 1, wherein the at least one processor is configured to:
select, from among the one or more first products, the one or more first products to be used for generating the one or more second products based on the evaluation result of the uncertainty; and
generate the one or more second products using the selected first products.

4. The information processing device according to claim 1, wherein the at least one processor is configured to evaluate the uncertainty using the evaluation value output by more than one of the one or more evaluators.

5. The information processing device according to claim 4, wherein the at least one processor is configured to select the one or more first products to be used for obtaining the one or more second products based on the evaluation value and a predetermined value.

6. The information processing device according to claim 5, wherein the at least one processor is configured to:
obtain a probability of excluding a first product based on the evaluation value for the first product; and
determine the one or more first products based on the probability.

7. The information processing device according to claim 5, wherein the at least one processor is configured to select the one or more first products to be used for obtaining the one or more second products based on weights and the evaluation value, the weights being based on the one or more evaluators.

8. The information processing device according to claim 1, wherein each of the one or more evaluators is a machine learning model.

9. The information processing device according to claim 8, wherein the machine learning model is installed in another information processing device.

10. The information processing device according to claim 1, wherein the at least one processor is configured to repeatedly execute the obtaining of the one or more first products, the evaluating of the uncertainty, and the obtaining of the one or more second products.

11. The information processing device according to claim 1, wherein each of the one or more first products is a crystal structure.

12. The information processing device according to claim 11, wherein the at least one processor is configured to execute Crystal Structure Prediction (CSP).

13. The information processing device according to claim 11, wherein each of the one or more evaluators is a model using a Neural Network Potential (NNP) method, and the evaluation value indicates at least energy of the first product or force of the first product.

14. The information processing device according to claim 11, which provides services in a form of Software as a Service (SaaS).

15. The information processing device according to claim 1, wherein the at least one processor is configured to:
receive a request from another information processing device; and
start, after receiving the request, execution of the obtaining of the one or more first products, the evaluating of the uncertainty, and the obtaining of the one or more second products.

16. An information processing system comprising:
at least one first information processing device configured to:
obtain one or more first products,
evaluate uncertainty of an evaluation value for each of the one or more first products using information from one or more evaluators, and
obtain one or more second products based on an evaluation result of the uncertainty; and
at least one second information processing device configured to:
receive a calculation request from a user, and
send an execution request to the at least one first information processing device in accordance with the calculation request, wherein
the at least one first information processing device starts the execution of the obtaining of the one or more first products, the evaluating of the uncertainty, and the obtaining of the one or more second products, after receiving the execution request from the at least one second information processing device.

17. An information processing method comprising:
obtaining, by at least one processor, one or more first products;
evaluating, by the at least one processor, uncertainty of an evaluation value for each of the one or more first products using information from one or more evaluators; and
obtaining, by the at least one processor, one or more second products based on an evaluation result of the uncertainty.

18. The information processing method according to claim 17, wherein the obtaining the one or more second products comprises:
determining, by the at least one processor, validity of each of the one or more first products based on the evaluation result of the uncertainty;
selecting the one or more first products determined to be valid as one or more valid products; and
determining the one or more valid products to be the one or more second products.

19. The information processing method according to claim 17, wherein the obtaining the one or more second products comprises:
selecting, by the at least one processor, from among the one or more first products, the one or more first products to be used for generating the one or more second products based on the evaluation result of the uncertainty; and
generating, by the at least one processor, the one or more second products using the selected first products.

20. The information processing method according to claim 17, wherein the evaluating the uncertainty comprises evaluating, by the at least one processor, the uncertainty using the evaluation value output by more than one of the one or more evaluators.

21. The information processing method according to claim 20 further comprising selecting, by the at least one processor, the one or more first products to be used for obtaining the one or more second products based on the evaluation value and a predetermined value.

22. A non-transitory computer readable medium storing a program that causes at least one processor to perform information processing comprising:
obtaining one or more first products;
evaluating uncertainty of an evaluation value for each of the one or more first products using information from one or more evaluators; and
obtaining one or more second products based on an evaluation result of the uncertainty.
